(19) United States
(12) Patent Application Publication
(10) Pub. No.: US 2002/0091306 A1
(43) Pub. Date: Jul. 11, 2002

(54) SHAPE MEMORY AND SUPERELASTIC IRIS RETRACTORS

(76) Inventors: EUGENE DE JUAN JR., PHOENIX, MD (US); ANDREW N. LAMBORNE, DENVER, CO (US)

Correspondence Address: Dike, Bronstein, Roberts & Cushman, EDWARDS & ANGELL, P.O. Box 9169, Intellectual Property Practice Group, Boston, MA, 02209, US

(21) Appl. No.: 09/234,621

(22) Filed: Jan. 21, 1999

Related U.S. Application Data
(60) Provisional application No. 60/072,844, filed on Jan. 22, 1998.

(51) Int. Cl. A61F 9/00

(52) U.S. Cl. 600/235

(57) ABSTRACT

A superelastic or shape-memory iris retractor for use in operating on the eye of a living creature, by means of which the iris is drawn outwards for widening the pupil. The iris retractor essentially comprises a superelastic or shape-memory grasping element and a preferably retainer which suitably is displaceable relative to the grasping element. In use, the grasping element is inserted suitably through a scleral, limbal or other incision and engages the iris for mechanically dilating the pupil. When removed, the grasping element can straighten so as not to catch on the scleral incision. In the shape-memory embodiment, the grasping element will revert to the element's original shape condition upon heat treatment. Both embodiments can withstand autoclaving for multiple reuses.

This application is a continuation of copending U.S. provisional application Serial No. 60/072,844, filed Jan. 22, 1998, which is incorporated herein by reference in its entirety.

### BACKGROUND OF THE INVENTION

1. Field of the Invention

The present invention relates generally to a surgical instrument adapted for retraction of an iris during operation on the eye of a living creature. More particularly, the invention concerns an iris retractor with a superelastic or shape-memory grasping element for retracting the iris and preferably on which is arranged a retainer element for fixing the iris retractor in a position with the iris retracted toward the corneal-scleral transition, or limbus.

2. Background

During eye surgery, the pupil often must be dilated, i.e. the pupillary diameter must be increased, to provide adequate viewing of the vitreoretinal portions of the eye. Adequate dilation of the pupil is essential, for example, during cataract and posterior segment surgery. Pupil dilation may also be required to extract foreign bodies lodged behind the inferior iris. In some situations however, a patient's pupil may resist dilation efforts. For example, in patients suffering from exfoliation syndrome, the pupils dilate submaximally. Past surgery or recent trauma, as well as other various physiological aspects, also may prevent the pupil from dilating adequately.

Several approaches have been reported to enhance pupil dilation. Some of these are principally pharmaceutical, others are surgical (i.e. incisional), and others utilize mechanical manipulation of the iris.

Pharmaceutical approaches are generally less preferred, primarily because in a significant number of patients, the pharmaceuticals simply do not work.

Surgical approaches that have been employed, including sphincterotomies and sector iridectomies, are also generally undesirable because of their cosmetic impacts as well as surgical complications due to bleeding. An iridectomy also produces the undesirable release of pigments into the eye. Another approach is to use sutures to tack the iris through the scleral wall, but the procedure requires quite delicate surgery and is time consuming.

Intraocular tacks also have been employed. In that approach, a stainless steel, disposable tack is provided with a hook built onto its proximal end, with a sharp anterior blade at the distal end. The blade is pushed through the peripheral cornea of the eye at the limbus by an introducer, and the hook is engaged to the iris, and subsequently pushed until the blade is imbedded into the corneal-scleral transition from the inside, tacking the iris in a dilated position. The introducer is then withdrawn leaving the tack in situ. This approach has the disadvantage of requiring insertion of the instrument essentially “blind” into the eye beneath the iris. As a result, the sharp leading edge of the instrument may unintentionally penetrate the iris, which can result in undesirable pigment release into the eye. Further, the tacks from time to time do not hold, causing substantial complications during a surgical procedure.

A recent approach, which is gaining in popularity, involves the use of translimbal iris hooks. See, e.g., Fuller et al., Am. J. Ophthal., 110:577 (1990) (“Fuller et al.”), which reports a certain iris retractor having a 0.3 mm diameter stainless steel wire hook fitted with a solid silicone stop. In use, the hook is inserted into a limbal incision of approximately 0.5 mm where it is able to grasp the iris and draw it back. The silicon stop is then adjusted to rest against the outer surface of the eye above the limbal incision and retain the hook in the drawn position. However, this device is awkward in use, as discussed more fully infra.

It would be desirable to devise improved devices and methods for iris dilation.

### SUMMARY OF THE INVENTION

The present invention provides a reusable iris retractor which allows precise handling during insertion and removal. The iris retractor utilizes a grasping element, e.g. a hook, comprising a shape-memory or superelastic material, preferably a nickel-titanium alloy.

We have found that the surgical instrument of the invention exhibits substantial performance advantages.

For example, in one aspect of the invention, the surgical instrument comprises a superelastic grasping element that allows for easy withdrawal of the instrument from a limbal or scleral incision, reducing trauma to the incision and the need for sutures.

Additionally, in another aspect of the invention, the surgical instrument comprises a shape-memory grasping element which can be adjusted before insertion according to the needs of the surgeon, or straightened out during removal from a limbal or scleral incision, but will regain its original shape upon being heated in an autoclave. This shape-memory feature enables extensive reuse of the device of the invention, thereby providing substantial cost and waste savings.

The invention further provides a grasping or hook element made from superelastic or shape-memory material, particularly a nickel-titanium alloy. Particularly preferred alloys are the nitinol family of nickel-titanium alloys. Other aspects of the invention are disclosed infra.

### BRIEF DESCRIPTION OF THE DRAWINGS

FIG. 1 depicts an eye shown diagrammatically and in cross-section, in which one region of the iris is retracted with a preferred iris retractor according to the invention.

FIG. 2 depicts a preferred reusable iris retractor of the invention.

FIG. 3 consists of FIGS. 3A-3F that show various views of a preferred iris retractor and elements thereof.

### DETAILED DESCRIPTION OF THE INVENTION

The invention provides substantially improved iris retractors. Thus, for example and as discussed above, iris retractors of the invention enable a surgeon to adjust the grasping element, e.g. a substantially U-shaped hook, prior to insertion into the limbal incision. Retractors of the invention also enable facile withdrawal of the grasping element without trauma to eye tissues. Retractors of the present invention also have the added benefit of the grasping element regaining its original shape after heat sterilization and maintaining its elasticity after repeated heat-sterilization cycles.

The retractors of the invention exhibit substantial advantages over prior devices. For example, a major disadvantage with the device reported in Fuller et al., supra, is the general inability to position the device's hooks and retain those hooks as desired for an extended period without lacerations to the eye. Another disadvantage with the Fuller et al. device is that during withdrawal of the hook, the relatively inelastic stainless steel wire may catch on the limbal incision into which the hook was passed. This is a particular problem because such injury may necessitate suturing, which can result undesirable post-surgical astigmatism. Additionally, to reuse the Fuller et al. device, the stainless steel wire must be manually reshaped, which after repeated cycles may result in breakage. Thus, the device typically can be employed only for a limited number of surgical procedures before it must be discarded.

One prior approach to the problem of the stainless steel's relative rigidity is to fashion the hook from medical grade nylon monofilament. One such hook is marketed by SURGIDEV Corporation. A major disadvantage with this approach is that the nylon element significantly degrades during autoclave sterilization, such that typically only one to three uses are possible before the hook must be discarded.

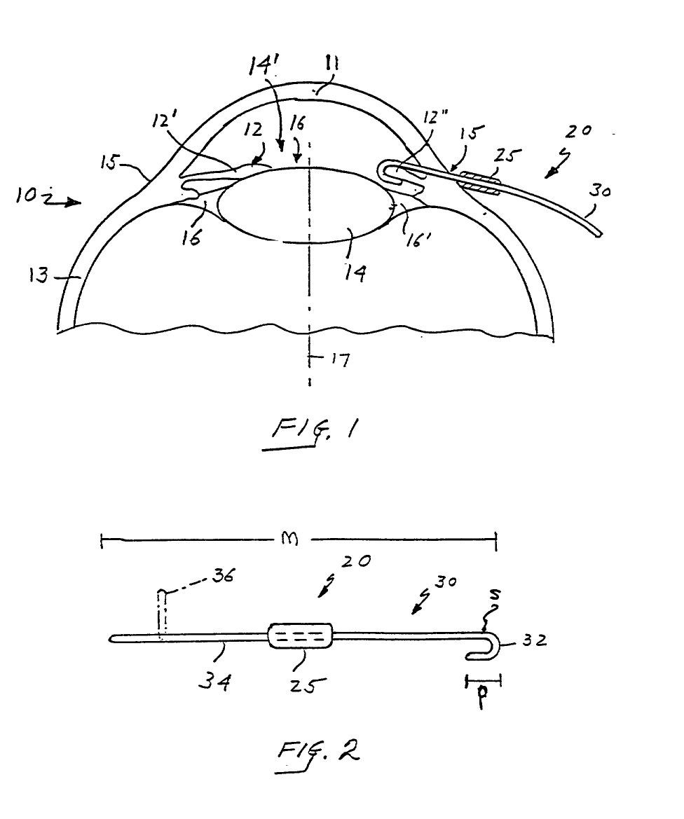

Referring now to the drawings, FIG. 1 shows an eye 10 in diagrammatic cross-section. The various structures of the eye are labeled: the cornea 11, the iris 12 with the regions 12′ and 12″, the sclera 13, the lens 14, the pupil 14′ and the zonulas 16, 16′.

As seen in FIG. 1, an iris retractor 20 engages region 12″ of the iris 12, which is drawn outwards relative to the line of vision 17 of the eye 10 and is held approximately at the limbus 15, or the transition between the cornea 11 and the sclera 13. The iris retractor 20 comprises a stop or clamping part 25, and a body part 30, which has a grasping or hook element end which passes through the cornea 11 and engages the iris region 12″.

FIG. 2 shows an illustrative embodiment of a preferred iris retractor of the invention. The retractor 20 comprises a body part 30, which at a distal end is bent into a grasping element 32, preferably a hook-shaped element as generally depicted in FIG. 2. A stop or clamping part 25 is disposed preferably slidably on a straight portion 34 of the body part. Optionally, a bent portion 36 may provide a gripping portion.

According to a first embodiment of the invention, preferably a major portion (e.g. at least about 20%, 30%, 40, or 50% of total mass) of body part 30 is formed of a superelastic material, preferably an alloy, particularly a nickel-titanium alloy. Alternatively, only a substantial portion of the grasping element 32 may be formed of such a superelastic material, with the remainder of the body portion formed of another material, such as stainless steel or plastic. By stating that “a substantial portion” of the grasping element is formed of the superelastic memory material, it is meant that at least about 50%, 60%, 70%, 80%, 90% or 95% of the length of the grasping element is formed of the superelastic memory material. Preferably, the entire device body 30 is formed of the superelastic material.

The superelastic material suitably will be durably (i.e. no breakage) elastic in dimensions of about 0.010″ or less and maintain this durable elasticity after repeated heat sterilization cycles, e.g. at least about 5, 10, 20 or 30 heat sterilization cycles. More preferably, the material should maintain durable elasticity in a wire having a diameter of approximately 0.005″ or less and maintain this elasticity after repeated heat sterilization cycles, preferably at least about 5, 10, 20 or 30 heat sterilization cycles. Typical sterilization temperatures are at least about 100° C., more typically at least about 120° C., 140°, 150° C. or 160° C. Autoclaving is often conducted at about 150° C. or 160° C.

Preferred materials for use as the superelastic material will exhibit a superelastic recoverably strain at least up to about 5% or 6%, more preferably up to about 7% or 8%, still more preferably a superelastic recoverably strain at least up to about 10%, 11% or 12%. Preferred superelastic materials also include those that exhibit a transformation fatigue life of at least about 50, 100 or 200 cycles at about 5% or 6% strain; at least about 10⁴ or 10⁵ cycles at 2% strain; and at least about 10⁶ or 10⁷ cycles at 0.5% strain.

A particularly preferred superelastic material for use to form a body portion 30 or grasping element 32 of a surgical instrument of the invention is the family of nickel-titanium alloys known as nitinol. Nitinol is commercially available from a number of sources, such as Special Metals Corporation (New Hartford, N.Y.) or Raychem Corporation. Nitinol is generally recognized as having a norminal composition of approximately 50% Ni and 50% Ti, although other amounts of Ni and Ti can be employed, e.g. an alloy containing about 55% Ni and 45% Ti.

For preferred alloys of devices of the invention, particularly nickel-titanium alloys such as nitinol, the quality of superelasticity can be characterized as the reversible and non-linear elastic deformation obtained when the alloy in its austentic state is strained at a temperature above the martensite-austenite transition, but below some maximum temperature at which martensite-austenite transition may be induced by stress. A nitinol alloy exhibits two stable phases—martensite and austenite—which correspond to a lower and a higher temperature respectively. Nitinol may be formulated with different percentages of nickel to select the temperature ranges for each of those phases and the transition temperature between martensite and austenite.

According to a second embodiment of the invention, preferably a major portion of the (e.g. at least about 20%, 30%, 40, or 50% of total mass) of body part 30 is formed of a shape-memory material, preferably an alloy, particularly a nickel-titanium alloy. As known in the art, shape memory is a physical property by which a plastically deformed material reverts to its original shape upon a change of temperature, usually heating.

As with the first embodiment, suitably only a substantial portion of the grasping element 32 may be formed of the shape memory material with the remainder of the body portion formed of another material, such as stainless steel or plastic. As with the first embodiment, stating that “a substantial portion” of the grasping element is formed of the shape memory material, it is meant that at least about 50%, 60%, 70%, 80%, 90% or 95% of the length of the grasping element is formed of the shape memory material. Preferably, the entire device body 30 is formed of the shape memory material.

Preferred materials for use as the shape memory material will exhibit a shape memory recoverable strain of between about 5.0% and about 9.5%, more preferably between about 6.5% and about 8.5%.

A particularly preferred shape memory material for use to form a body portion 30 or grasping element 32 of a surgical instrument of the invention is the above-discussed family of intermetallic nickel-titanium alloys known as nitinol.

As discussed above, nitinol exhibits two stable phases of martensite and austenite. The martensite phase is malleable, such that the material may be bent to achieve any shape, which it will retain, i.e. its “memorized” shape. Upon deforming a nitinol element in accordance with the invention and then heating the element to a temperatures above the martensite-austenite transition temperature where the austenite phase is dominant, the element will revert to its original “memorized” shape.

During formation of a shape memory device, e.g. a wire, the shape to be remembered is formed, mechanically maintained, and the wire is heated far above the martensite-austenite transition temperature and then cooled. Upon cooling into the martensite range, the shape is memorized, however the wire is now easily deformed to change shape. Upon heating, the wire will revert to the memorized shape.

The shape-memory embodiment is malleable so that a surgeon can bend it into a different shape when necessary, but autoclaving returns the retractor to its original shape. Again, the nitinol will endure many cycles of use and autoclaving or other heat treatment. While autoclaving or other heat treatment is needed to reform the original element shape, other sterilization methods also may be employed such as an ethylene oxide (ETO) bath or the like.

By stating that device returns to its original shape in the shape-memory embodiment, the device may suitably return to within about 30%, more preferably to within about 20%, 10% or 5% of its original shape, i.e. within such percentages of the device's original length, radius of curvature in the case of a curved feature, etc. Preferably, the device returns to within less than about 5% of its original shape, or returns to essentially as, or the same as, its original shape.

Suitable dimensions of retractor devices of the invention and the components thereof can vary rather widely and can be readily determined by those skilled in the art based on the present disclosure. In general, the device should have a shape and length so that the device is capable of being inserted into a patient for use in eye surgical procedures. For many applications, the length of device 20 (length m in FIG. 2) suitably may be from about 0.2 inches to about 0.7 inches, more typically from about 0.25 to about 0.5 inches.

Suitable diameters of the body portion 30 are from about 0.001″ to about 0.012″, although again other diameters could be employed. The length of grasping element (length p in FIG. 2) is preferably from about 0.020 to about 0.050 inches. The grasping element is preferably curved-shaped, with an outer diameter (i.e., distance from the outer edge of parallel portions of curved-shaped element) from about 0.01 inches to about 0.030 inches, more typically an outer diameter from about 0.01 inches to about 0.020 inches. A grasping element that has a curve of from about 160° to about 210° is generally preferred, and more preferably is a curve of about 180° as generally depicted in FIG. 2. Preferably, a retainer element (if present) has a disc-shape with a diameter of from about 0.050 inches to about 0.090 inches and a thickness of from about 0.008 inches to about 0.02 inches.

A preferred size for the grasping element is a wire between about 0.0020″ and 0.0050″ in diameter, more preferably a diameter of between about 0.0025″ and 0.0040″. A thinner wire may not be sufficiently stiff to push through the incision, and a thicker wire may be too stiff to allow the grasping element to deform as desired upon withdrawal of the element during surgery.

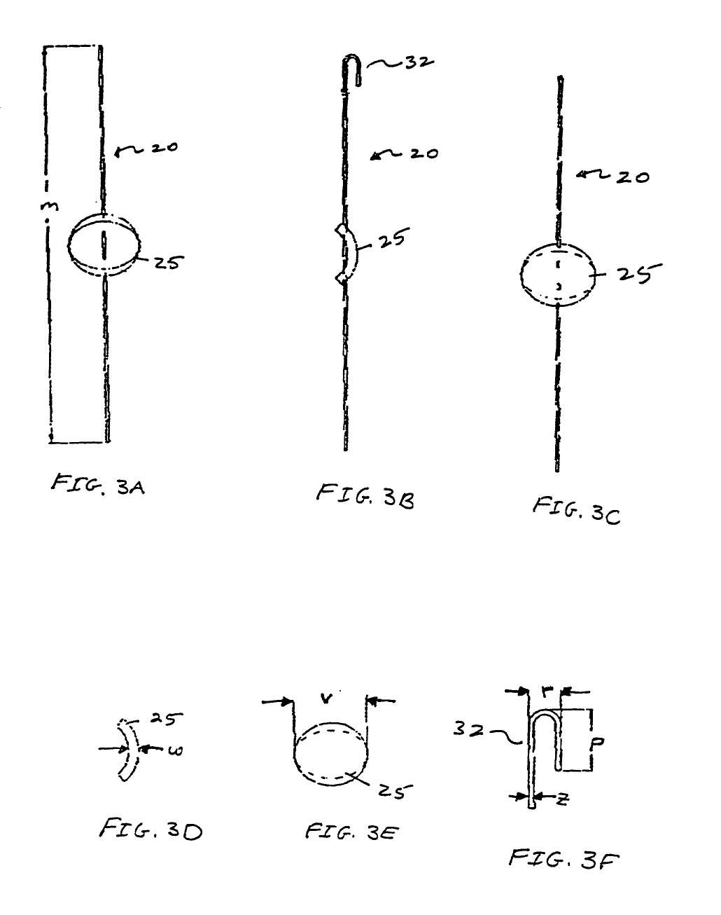

A particularly preferred retractor device of the invention is of the configuration shown in FIGS. 3A-3F wherein the usable length of the device 20 (length m) is 0.375 inches; grasping element is 0.030 inches long (length p in FIG. 3F) with an outer diameter (diameter r in FIG. 3F) of 0.015 inches and has a U-shaped curve of about 180° and a radius of curvature of 0.018″; and stop or retainer element 25 is slidably disposed at about the middle of the retractor and is disc-shaped as depicted in FIGS. 3A-3F with a diameter (diameter v in FIG. 3E) of 0.070 inches and a thickness (thickness w in FIG. 3D) of 0.012 inches. In that preferred embodiment, preferably the entire device body is formed of nitinol wire having a diameter (z in FIG. 3F) of 0.0025 inches, and the stop element is formed of silicone.

The end of the grasping element 32 preferably will not be sharpened to a point to avoid damage to the iris or other delicate tissues in the eye. If the wire is formed from mechanically cut lengths resulting in a sharp edge, the end of the grasping element may be mechanically smoothed or laser or arc welded to form a smooth rounded end.

In iris retractors of the invention corresponding to the device shown in FIGS. 2 and 3, the body portion 30 can be suitably constructed using an alloy wire and small cylinders of silicone punched from a sheet to form the retainers or stops 25. A needle is punched through the center of the cylinder, and the annealed wire is threaded into the needle. The needle can then be removed so the silicone stop 25 is riding on the straight portion 34 of the retractor, as seen in FIGS. 2 and 3. The stop 25 is preferably positioned between the grasping element and distal portion of the retractor. The stop element can be affixed to the device by a variety of other methods, such as molding silicone directly onto the wire forming the device body portion, preferably over a sufficiently large area of the wire so that undesired slippage of the stop is avoided.

As mentioned above, suitable superelastic and shape-memory materials are commercially available, such as nitinol which is available in annealed as a straight wire form. To form a desired grasping element, such as a U-shaped hook portion, the wire can be constrained in the desired shape to provide the grasping element and heated to annealing temperature, approximately 400° C. or 500° C. depending on the nitinol alloy that is employed. Nitinol can be processed differently during manufacture, e.g. distinct annealing treatments, to selectively provide either superelastic or shape-memory properties.

Both the first and second embodiments of the invention may be employed in substantially the same manner in the course of a surgical procedure, although they have different properties which might make one or the other preferable for a particular intended use. In one typical procedure using a device of the invention, a limbal incision is first made. Preferably the incision is a self-sealing stab incision. The instrument of the invention is then passed, grasping element first, into the incision until the grasping element can be looped over the edge of the iris. The body portion still extending from the incision is drawn back, and the instrument is held in the drawn position by sliding the silicone stop toward the incision until it contacts the surface of the eye over the limbal incision. When the related procedures requiring dilation are finished, the reverse procedure is carried out. Procedures for use of iris retractors in general also have been disclosed in U.S. Pat. No. 5,174,279.

When using the shape-memory embodiment, the surgeon may adjust the shape of the grasping element prior to insertion into the incision. Because the material chosen is malleable at ambient temperature, the grasping element—or if the entire body portion were made of the shape-memory material, the body—is readily deformed at the surgeon's convenience. Upon withdrawal of the instrument, there is no danger of trauma to the incision from the inside of the eye as with stainless steel hooks because the grasping element may deform to exit the incision without injury to the patient. Upon autoclaving, the instrument will regain its original shape and be ready for reuse. Upon cooling, the instrument is again malleable to be adjusted according to the surgeon's preference.

When using the superelastic embodiment, the surgeon may use the instrument similarly as the prior nylon filament devices. After each use, the instrument may be heat sterilized, for example in an autoclave, and used virtually indefinitely, at least until the silicone stop degrades. At that point, the instrument may be remanufactured or simply discarded. This is a significant advantage over nylon monofilament devices that are generally limited to about 1 to 3 uses.

Typical patients for use of the devices of the invention are mammals, particularly humans, such as a mammal undergoing cataract or posterior segment surgery, or a mammal requiring removal of a foreign object lodged in the eye. In addition to humans, devices of the invention also will have substantial veterinary applications, particularly for domesticated animals such as dogs and cats.

The invention also includes device kits which comprise one or more the surgical instruments of the invention preferably supplied in a sealed package in a sterile condition.

All documents mentioned herein are fully incorporated herein by reference.

Although the invention has been shown and described with respect to exemplary embodiments thereof, various other changes, additions and omissions in the form and detail thereof may be made therein without departing from the spirit and scope of the invention.

What is claimed is:

1. An iris retractor suitable for use when operating on an eye of a living creature, said iris retractor comprising:
a body portion having a proximal and a distal end;
a grasping element at the distal end of said body portion for retracting an iris, wherein a substantial portion of the grasping element is formed of a shape-memory material.

2. An iris retractor of claim 1 wherein the body portion is formed of a shape-memory material.

3. An iris retractor of claim 1 or 2 wherein the shape-memory material is a nickel-titanium alloy.

4. An iris retractor of claim 3 wherein the shape-memory material is nitinol.

5. An iris retractor suitable for use when operating on an eye of a living creature, said iris retractor comprising:
a body portion having a proximal and a distal end;
a grasping element at the distal end of the body portion for retracting an iris, wherein a substantial portion of the grasping element is formed of a superelastic material.

6. An iris retractor of claim 5 wherein the body portion is formed of a superelastic material.

7. An iris retractor of claim 5 or 6 wherein the superelastic material is a nickel-titanium alloy.

8. An iris retractor of claim 7 wherein the superelastic material is nitinol.

9. An iris retractor of any one of claim 1-8 wherein a retainer element is positioned disposed on the body portion between said proximal and distal ends thereof.

10. A surgical instrument comprising:
a body portion having a grasping element; at least a portion of the grasping element comprised of a shape-memory material.

11. A surgical instrument of claim 10 wherein said shape-memory material is a nickel-titanium alloy.

12. A surgical instrument of claim 10 wherein the shape-memory material is nitinol.

13. A surgical instrument comprising:
a body portion having a grasping element; at least a portion of the grasping element comprised of a superelastic material.

14. A surgical instrument of claim 13 wherein said superelastic material is a nickel-titanium alloy.

15. A surgical instrument of claim 13 wherein said superelastic material is nitinol.

16. A method for retracting the iris of a living creature, comprising the steps of:
(a) providing an iris retractor comprising:
a body portion having a proximal and a distal end,
a grasping element at the distal end of the body portion for retracting an iris, wherein at least a substantial portion of the grasping element is formed of a shape-memory material;
(b) inserting the grasping element of the iris retractor into an incision on the eye;
(c) grasping the iris with the grasping element and withdrawing the iris toward the incision;
(d) releasing the iris and removing the iris retractor from the incision; and
(e) subjecting the iris retractor to a temperature change such that said grasping element substantially regains its original shape.

17. The method of claim 16 further comprising adjusting the shape of the grasping element prior to inserting the grasping element into the patient.

18. The method of claim 16 wherein the grasping element is comprised of nitinol.

19. A method for retracting the iris of a living creature, comprising the steps of:
(a) providing an iris retractor comprising:
a body portion having a proximal and a distal end;
a grasping element at the distal end of the body portion for retracting an iris, wherein at least a substantial portion of the grasping element is formed of a superelastic material;
(b) inserting the grasping element of the iris retractor into an incision on the eye;
(c) grasping the iris with the grasping element and withdrawing the iris toward the incision;
(d) releasing the iris and removing the iris retractor from the incision.

20. The method of claim 19 further comprising sterilizing said iris retractor by heat; and reusing and resterilizing said iris retractor a plurality of times.

21. The method of claim 19 wherein the iris retractor is sterilized and reused at least five times before discarding the device.

22. The method of claim 19 wherein the grasping element is comprised of a nickel-titanium alloy.

23. The method of claim 19 wherein the grasping element is comprised of nitinol.

24. A surgical device kit comprising at least one iris retractor of any one of claims 1-9 packaged in sterile condition.

25. A surgical device kit comprising at least one surgical instrument of any one of claims 10-15 packaged in sterile condition.
